(19) United States
(12) Patent Application Publication
(10) Pub. No.: US 2021/0045286 A1
(43) Pub. Date: Feb. 18, 2021

(54) Self-Regulating Smart Irrigation System

(71) Applicant: Ramesh Gopalan, Fremont, CA (US)

(72) Inventor: Ramesh Gopalan, Fremont, CA (US)

(73) Assignee: Ramesh Gopalan, Fremont, CA (US)

(21) Appl. No.: 16/994,594

(22) Filed: Aug. 16, 2020

Related U.S. Application Data
(60) Provisional application No. 62/888,392, filed on Aug. 16, 2019.

(51) Int. Cl. A01C 23/04; G01N 33/24; A01G 25/16

(52) U.S. Cl. CPC A01C 23/042; G01N 2033/245; A01G 25/167; G01N 33/246

(57) ABSTRACT

A self-regulating irrigation system to provide water and dissolved nutrients to plants through a tubing network is described including a sensor actuator circuit comprising an sensing circuit which senses aspects of soil condition through sensing a voltage between sensing terminals in the soil. Aspects of soil condition include moisture, mineral content and acidity or pH level. The sensed voltage is input to a comparator circuit whose output becomes input to an actuator circuit to activate a valve to supply a corresponding branch of the network with desired water or fluid nutrient.

This application claims benefit from the provisional patent application 62/888,392 filed Aug. 16, 2019 by the present inventor.

### BACKGROUND OF THE INVENTION

Technical field: This invention pertains to controlling systems for irrigation networks providing water and nutrients to plants.

About 80% of water usage is for agriculture and farming, to grow the plants that are primary food source for all humanity. In effect, humans consume more water through the food they eat than they consume for drinking, bathing, cleaning home, gardening and making all the goods used in the modern industrial economy. In a world facing unpredictable climate change, hot, dry areas are getting hotter and drier, with increasing variability in precipitation and weather patterns as seen in increased incidence of both drought and floods, often in the same geographical areas within the same year. Many areas of the world, from the American South West to the Middle East and India face a future of increased water stress, with expected demand exceeding an unpredictable supply. Ground water aquifers that were stable for millenia are found to be rapidly depleted within a generation.

It is clear that the water-poor future of the world will need to address optimizing water use by its largest consumer—agriculture. Drip irrigation, which delivers only water to the root zone of growing plants, avoiding wastage through evaporation over wider areas, is a key technology to enable this future.

Drip irrigation expanded particularly from Israel in the 1960s with the availability of low cost polyethylene or plastic tube and drip accessories including valves and emitters. The concept is simple: the water is provided from a point or linear source where water is provided only to the root zone of plants where it is readily absorbed into plant tissue while minimizing waste of water to unneeded areas and loss through evaporation.

The technology is simple but is able to reduce water usage by up to 90% to let the ‘desert bloom’ and recently shown to grow even water-thirsty crops like rice in the arid, desert areas of the Middle East.

More recently, the availability of sensors, such as for soil moisture, or acidity, have transformed the drip irrigation landscape yet again, bring it into the world of Big Data and the Cloud. These have become available from a variety of vendors including Netafim of Israel, Jain Irrigation of India, with subsidiaries all over the world, including in the USA. Most often the main input is weather conditions, but frequently now soil moisture is measured and beamed through wired or wireless networks to drones, satellites to be analyzed into maps stored in the cloud. Central control stations then respond by opening valves to provide watering to areas that have become too dry. The feedback loop, although effective, is complicated, and depends on multiple steps—the soil conditions are measured by a point sensor; this information is transmitted to a controlling computer which makes a decision on whether or not to open a valve to provide water or other nutrient to the measured target area; and then the decision is transmitted—usually by a wire to the valve that controls the flow of water to the target area. This requires multiple wireless networks to collect the data to servers that store the measured data and software that makes decisions upon them; wireless networks to send back the decision based on the data, and, also, power cables to actuate the valves that flow water or nutrient to the target areas that need it. There are also sensors, such as from Rainbird, which use electromagnetic tomography of a wide area to sense moisture content. These are a complicated technology, and costing over $100 each, are not accessible to the small-plot farmer that is the future of agriculture, particularly in Asia and Africa. The key challenge is that they are based on analysis of data collected over a wide area, and not sensitive to or accurately gathering the information around the root zones of the crops.

The current art can involve complicated, multi-layered technologies, with multiple points of failure, for the simple task of watering a plant whose root zone has become too dry. Agriculture, humankind's most ancient occupation, should not dependent on wireless communication networks, decision-making programmed into computers, and wired power lines which can breakdown frequently, particularly in remote arid areas, in farms spread over thousands of acres in the developed world. Meanwhile, most farmers in Asia and Africa are small-plots farmers who may not be able to see benefit or be to afford the smart sensing technologies.

The need is for a simple, easy-to-understand-and-operate system of providing irrigation targeted only to the root zone of plants which is self-regulating, self-powered, optimizing the usage of water without the need for complicated multi-layered technologies.

### DESCRIPTION OF THE INVENTION

The invention is a self-regulating, self-powered system for providing water and nutrient to control and optimize soil conditions in the vicinity of the root zone of growing plants.

Soil conditions such as moisture level, acidity—pH, mineral and nutrient content, are measured through sensors that transduce these parameters into a voltage. This sensed voltage, representing the parameter of interest, is then compared with a reference voltage which represents the ideal or target value of the parameter of interest. The sensed parameter is compared with the reference parameter by a voltage comparator circuit, and if the parameter value is sufficiently different from the target or reference value it activates a transistor switch to turn on and open a fluid valve, supplying the needed water or nutrient to the targeted soil area. The fluid valve may be on a parallel branch of pipe or tubing circuit in an irrigation network supplied with water, fluid nutrients or liquid fertilizer.

The sensor-actuator controlling circuit may be understood as being comprised of two connected parts—a) the soil sensor circuit that drives the action of b) the valve actuator circuit. The soil sensor circuit senses the soil condition, typically as a voltage across two sensing terminals and compares this sensed voltage against a reference voltage, typically through an electronic circuit component such as a comparator. The output of this comparator is then supplied as input to the actuator portion of the sensor-actuator circuit which opens or closes a fluid valve depending the relative values of the compared inputs to the sensor circuit. The voltage comparator, examples including the LM393 series from Texas Instruments, provides a binary output—ON or OFF—depending on if the sensed voltage is above or below the reference voltage. This output of the comparator may be input to a electronic switch which, for example, could be a MOSFET transistor in n-channel depletion mode—which is activated to the ON state with a sufficiently high positive voltage supplied to the gate, allowing a current to flow between the transistor source and drain. Such a transistor or electronic switch, when in series with an solenoid fluid valve, will actuate this (normally-closed) valve to open (or close) by supplying (or shutting off) the needed current to operate it.

The moisture content of soil may be sensed by measuring the resistance as the sensed voltage between two sensing terminals with a given reference current flowing between them. If the soil becomes too dry then its resistance increases, as does the voltage across the sensing terminals. When this sensed voltage exceeds a reference value, such as provided by the same reference current flowing through a reference or adjustable resistor of known value, then the comparator in the circuit will output an ON voltage signal to the transistor or electronic switch, causing it to conduct current. This will allow current to flow through the valve from the power supply, actuating its operation and allowing water or other fluid nutrients to flow to the network branch or loop corresponding to the location of the sensing terminals.

As an example, typical garden soil will, when fully saturated with water, measure a resistance of less than 5 kiloOhms (kΩ) between two rod conductors, each of galvanized steel about 1/16″ in diameter, placed about 1 inch apart, with each penetrating about 1 inch into the soil. But when the soil is very dry the resistance will exceed 10 MegaOhms (MΩ) between the same two conductors. A 10 Volt supply, for example, will have a reference current of about 10 microAmps through a reference resistor of about 1 MΩ, if such a resistor is used to set the reference voltage. As the soil resistance between the reference terminals rises with drying soil from below a few kiloohm to above 1 megaOhm, the voltage will rise from nearly ground (or zero volts) to nearly the supply voltage of 10 volts.—this voltage change will be detected by the comparator and accordingly used to output a high voltage to the transistor switch, turning on the current to the solenoid valve supplying water. When the moisture content around the sensing terminals gets sufficiently high enough then the resistance will drop back below a few kilohms, which will conversely set the comparator to switch the voltage low and the transistor into the non-conducting state, causing the water supply through the solenoid valve to turn off.

A typical embodiment of the invention may use solenoid valves that are normally-closed NC and operate ON—opening the valve—when a voltage exceeding about 5V is supplied with current between 0.3-0.7 Amperes allowed to the valve's terminals.

The entire system may be powered by a solar panel providing around 12V and about 0.5 Ampere of current, although less is adequate for most practical cases.

Such a system may comprise of many such valves each coupled to its corresponding sensing terminals in the nearby soil. Each such valve may supply a parallel branch of an irrigation network so that even if the main supply to the network remains open each branch is supplied with water only when the corresponding soil area turns dry.

The reference voltage may be adjustable, such as with an adjustable resistor or potentiometer to target low or high moisture content in the soil. This allows for different plant types, requiring different moisture content levels, to be irrigated from the same network, while the corresponding sensors are each set to different reference resistor or voltage values.

The source of sensed and reference voltage can be due to other physical aspects that represent soil condition. For example, it may be mineral content or acidity. In such cases as well, the voltage between two sensing terminals will be representative of these parameters and may be used to supply minerals, nutrients through the irrigation network until the appropriate target conditions are achieved in soil around the sensing terminals.

It is well known that two dissimilar conductors in soil have an electrochemical potential that varies according to the acidity or alkalinity of the soil. This phenomenon can be used to target acidity of the soil within the desired range, supplying dissolved acids or alkaline supplements to the soil to target the voltage between the sensing terminals and hence the acidity or pH of the soil to within the desired range.

When a solar panel is used as the power supply for the controlling circuit of each valve it allows the irrigation network to be operated off the power grid. Most plants need watering in the morning, during the ‘light phase’ of photosynthesis. The solar panel would automatically enable this by operating the controlling circuit to open the corresponding valves only in the morning when the sun comes up, providing water to the corresponding branches or loops of the irrigation network only until they reach their corresponding target moisture content levels. The sensing terminals may be disposed in various geometrical formats in the soil, including to enable averaging of the soil parameter of interest over wide areas.

For example, one of the limitations of drip irrigation is that it typically uses only point or line sources of water to the soil. The supplied water slowly diffuses through the soil from the immediate vicinity of the drip zone to eventually reach the entire root zone of the plants being irrigated. In such cases, the drip points may be placed near the center of the root zone of the plants while the sensing terminals may be placed at the edges of the root zone, or ‘drip-line’ of trees. The water supply through the opened solenoid valve then continues until water from the center of the drip zone diffuses outward to the edges of the root zone where and when the sensing terminals, sensing adequate moisture throughout the root zone, will turn off the solenoid valve.

The comparator outputs from multiple sensing terminals which sense more than one location and and more than one aspect of soil condition may be combined in OR or AND logic to turn on the electronic switch to enable the solenoid supply valve to open to meet the target conditions.

A self-regulating, self-powered, off-grid system is thus described which can supply an irrigation network for diverse plant and soil types from a single main supply, without need for external intervention.

### DESCRIPTION OF THE DRAWINGS

FIG. 1 shows the operation of a typical embodiment of the invention as represented by the sensor-actuator circuit. This is the controlling circuit which will self-regulate the irrigation system by actuating the opening of the valve to supply water only when the soil condition—moisture content—presents a high enough resistance from dry soil.

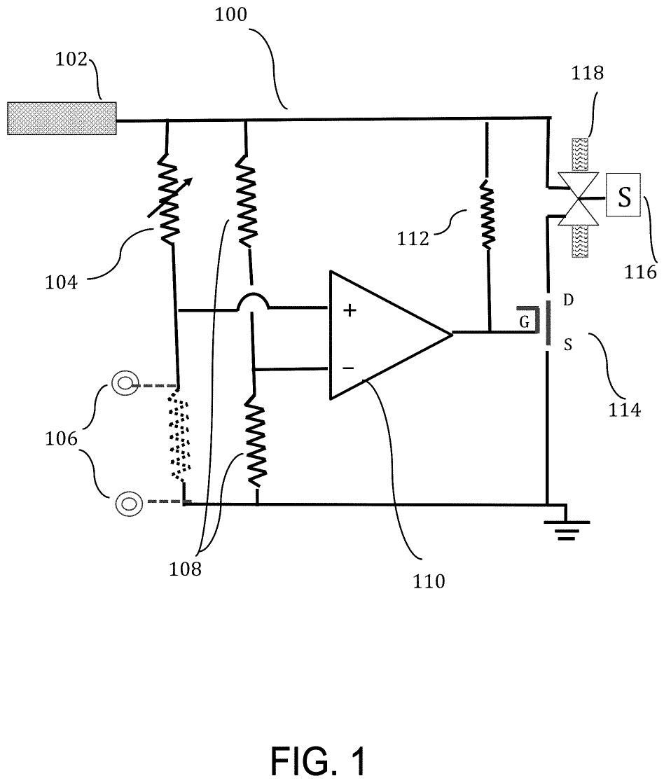

100—is the sensor-actuator circuit comprised of a soil sensor circuit section and a valve actuator section
102—is the electric power source—a solar panel or battery—providing 12 Volts supply to and about 0.5 Amperes to the sensor-actuator circuit in this embodiment
104—an adjustable resistor or potentiometer to be compared with the resistance across the sensing terminals
106—the sensing terminals: the sensed voltage is measured across the soil resistance between these terminals
108—the reference resistors—about 1 Megaohm in this embodiment—which set up the reference voltage to be compared with the sensed voltage at the comparator
110—the comparator or similar electronic component which compares the voltages at its two inputs to output a high or low voltage depending on the polarity of their relative difference.
112—a ‘pull-up’ resistor, about 1 kiloohm in this embodiment, that sets the voltage to the gate of the MOSFET to near the supply voltage.
114—MOSFET transistor in n-channel depletion mode
116—solenoid valve—normally closed NC—in this embodiment, typically requiring less than 0.5 A at 12V to open.
118—tube or pipe through the solenoid valve providing water and or fluid nutrients to the corresponding branch of the irrigation network.

### SUMMARY

A self-regulating irrigation system to provide water and dissolved nutrients to plants through a tubing network is described including a sensor actuator circuit comprising an sensing circuit which senses aspects of soil condition through sensing a voltage between sensing terminals in the soil. Aspects of soil condition include moisture, mineral content and acidity or pH level. The sensed voltage is input to a comparator circuit whose output becomes input to an actuator circuit to activate a valve to supply a corresponding branch of the network with desired water or fluid nutrient.

What is claimed is:

1. A self-regulating irrigation system providing water and nutrients to plants in soil comprising
an electric power source;
a plurality of valves with each such valve in fluid connection with a branch of an irrigation network;
each valve being electrically controlled by a sensor-actuator circuit comprised of an actuator circuit being coupled to the electronic output of a soil sensor circuit;
each soil sensor circuit being configured to measure a sensed voltage between sensing terminals disposed in adjacent soil;
each soil sensor circuit being further configured to determine the difference between sensed voltage and a reference voltage;
whereby the actuator circuit operates the corresponding valve to open or close as a function of an aspect of soil condition.

2. The soil sensor circuit of claim 1 comprising a comparator or operational amplifier

3. The actuator circuit of claim 1 comprising a field effect transistor

4. The aspect of soil condition of claim 1 being moisture content, nutrient or mineral content or pH level.

5. The sensed voltage of claim 1 being measured across the resistance between the sensing terminals with a given reference current from the power source flowing between them.

6. The sensed voltage of claim 1 being the electrochemical potential between the sensing terminals comprised of dissimilar conducting materials.

7. The power source of claim 1 being a solar panel.

8. The power source of claim 1 being a battery.

9. The sensing terminals of claim 1 being a plurality of conductors disposed in soil

10. The plurality of conductors of claim 6 being disposed in an array whereby sensed soil condition is averaged over area of array.

11. A plurality of the sensing terminals of claim 1 whereby the corresponding plurality of sensed voltages and one or more reference voltages may be combined through comparators in AND or OR logic as input to a transistor or electronic switch to actuate the operation of the corresponding valve.

12. A plurality of aspects of soil condition which may be combined in AND or OR logic to actuate the valve of claim 1.
